(19) United States
(12) Patent Application Publication
(10) Pub. No.: US 2025/0281724 A1
(43) Pub. Date: Sep. 11, 2025

(54) CATHETER INSERTION SYSTEMS

(71) Applicant: University of Pittsburgh - Of the Commonwealth System of Higher Education, Pittsburgh, PA (US)

(72) Inventors: William W. Clark, Wexford, PA (US); Cameron Dezfulian, Bellaire, TX (US); Brandon J. Barber, Pittsburgh, PA (US)

(73) Assignee: University of Pittsburgh - Of the Commonwealth System of Higher Education, Pittsburgh, PA (US)

(21) Appl. No.: 18/597,764

(22) Filed: Mar. 6, 2024

(51) Int. Cl. A61M 25/06

(52) U.S. Cl. CPC A61M 25/065; A61M 2205/3538; A61M 2205/3553

(57) ABSTRACT

Disclosed catheter insertion systems enable the user to identify the location of the needle based on the electrical properties of subcutaneous tissue relative the electrical properties of other fluids such as blood or air. Disclosed systems can include one or more of the following features: 1) the catheter assembly is modular (e.g., the catheter can be connected and disconnected from the detection unit at will); 2) the detection unit employs an electrical circuit that allows for the discernment between subcutaneous tissue and blood; 3) the system assists the end user with catheter advancement via an indicator output.

### ACKNOWLEDGMENT OF GOVERNMENT SUPPORT

This invention was made with government support under Grant No. P50FD006427 awarded by the Food and Drug Administration. The government has certain rights in the invention.

### FIELD

This disclosure is related to the field of catheter insertion systems.

### BACKGROUND

Many physicians use peripheral intravenous catheters (pIV) to administer lifesaving drugs in a rapid manner. Although millions of pIV's are placed on a daily basis around the world, approximately 25% of patients require multiple attempts to obtain a (pIV). Failure to correctly place an IV the first time increases the patient's pain and the cost associated with the procedure.

Peripheral vascular access is the mainstay of rapid drug delivery in modern healthcare and absolutely essential for inpatient/emergency room care and in certain outpatient settings. Yet placement of a peripheral intravenous catheter (pIV) to facilitate this is challenging despite a nearly universal mandate that all inpatients have a pIV in anticipation of medical emergencies. Approximately 25% of patients require multiple attempts to obtain a pIV resulting in a reported average of 2.2 attempts per successful pIV placement. When one considers the 1.2 billion pIV's purchased worldwide last year, this amounts to a staggering number of failures and a sizable problem within healthcare.

The cost of this difficulty is substantial. Failure to obtain peripheral vascular access subjects patients to increasing pain and distress such that most patients rate pIV placement as their most painful hospital procedure. Delayed pIV placement delays therapy (e.g. antibiotics, blood, resuscitation fluids and medications), which in a number of studies is linked to worsened clinical outcomes. The financial costs of failed pIV attempts are also substantial and largely stem from the personnel time involved with repeated attempts rather than the trivial (˜$2) cost of a catheter. In children, the median cost of pIV placement was $41 with 72% of the patients having success with 1 or 2 attempts. But this 72% accounted only for 53% of the costs. The other half came from the 28% of patients requiring ≥3 pIV attempts costing $69-125 for each patient. These personnel costs are not reimbursed. Insurers pay hospitals for a diagnostic code (e.g. pneumonia) that is structured to account for all costs associated with treating the diagnosed condition. If a nurse spends 30 minutes to place a pIV to give antibiotics the hospital reimbursement is no different than if that pIV had been placed within a minute. Failure to place pIV's results in escalation to midline or central venous access which carries greater risk to the patient, time for the practitioner, and cost to the hospital. More challenging IV placement also results in shorter dwell time (IV durability). Finally, difficulty in placing pIV results in higher rates of complications such as phlebitis, thrombosis, infection, and drug extravasation which in the most severe circumstances results in limb amputation. Furthermore, infiltration (medication leaking to surrounding tissue due to improper placement or dislodged catheter) is believed to be a common event that is massively under reported.

In most hospitals, pIV placement is performed primarily by nurses. Larger hospital systems often employ teams of IV nurses. These nurses are very good at prospectively identifying difficult pIV cases, and risk factors for “difficult” pIV access are well known and include pediatric, elderly, obesity, sickle cell anemia, IV drug abuse, prior hospitalizations, and diabetic patients. This is important in that nurses can therefore define which patients warrant use of a novel technology like the present invention to maximize benefits while minimizing costs.

As an example, a typical IV nurse team may be a 4-person group. If the team can identify the difficult patients and if a device were available that would allow insertions on the first attempt for these patients, then the IV teams could be reduced to 3 people.

Improving first-time pIV access and durability can result in reduction in patient pain and adverse events, improved outcomes, and reduced institutional legal risk and financial costs. Interviews were conducted with 50 hospital staff (including 32 nurses and 7 members of IV teams) to determine the root cause of failed pIV attempts. The number one reason was inability to advance the IV catheter after an initial blood flash in the needle (see the following section for how pIV's are currently placed) which the operators attributed to 1) passing through the vein by the time the flash was visualized, 2) glancing or tearing the vein with manipulations, and 3) being too shallow in the vein. Other issues identified included vein rolling, vein size too small or too fragile to support the catheter, and patient movement.

One solution to assist with catheter placement which has been in use since 1953 is the Seldinger technique, which involves the placement of a guidewire through a needle into the blood vessel or space where one wishes to place a catheter followed by catheter insertion over the guidewire. Since insertion of the flexible guidewire is less traumatic to the vessel and the size is smaller than the needle with a length far longer than the needle bevel, this reduces vessel trauma and improves success in catheterization. The Seldinger technique requires an elaborate sterile field. Integrated units which incorporate a guidewire contained in a sterile sheath, which sits within a traditional catheter over needle design, permits sliding in the guidewire followed by catheterization without the need for sterile setup. Such devices are currently in use for arterial and venous access. However, in the case of venous access these systems fail most often due to failure to recognize vein entry based on blood return or premature sliding of the guidewire resulting in infiltration.

There is therefore a need for improved detection and insertion systems, especially ones that can address the inability to recognize vein entry and to assist the user with timely advancement of a guidewire to facilitate catheterization within fragile or small veins thus addressing the primary deficiencies.

### SUMMARY

Disclosed are catheter insertion systems that enable the user to identify the entry of a percutaneously inserted needle, in some examples including an integrated guidewire, into a fluid or air-filled space, such as a blood vessel or pneumothorax, based on the electrical properties of the subcutaneous tissue relative the electrical properties of the fluid (e.g. blood) or air which are sensed by a current passed between the needle and guidewire. Various embodiments of needles and detection units are described. Disclosed systems can include one or more of the following features: 1) the catheter assembly is modular (e.g., the catheter can be connected and disconnected from the detection unit at will); 2) the detection unit employs an electrical circuit that allows for the discernment between subcutaneous tissue and various fluids such as blood, pleural or pericardial effusions or air; and 3) the system assists the end user with catheter advancement via an indicator interface. Modified versions of this system can also be employed to insert catheters into a variety of additional spaces where the needle passes first through subcutaneous fat and muscle before entering fluid or air. These include placement of thoracostomy catheters into pleural effusions or pneumothoraces, insertion of catheters into the cerebrospinal fluid or epidural space, insertion of arterial catheters, insertion of pericardiocentesis catheters into a pericardial effusion, insertion of a peritoneal catheter into peritoneal fluid and insertion of percutaneous tracheostomy tubes.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 shows standard pIVs in clinical use. pIVs are arranged from 14 (left) to 24 (right) gauge. The catheter sits over the needle a small distance back from the hub.

FIG. 2 illustrates a Seldinger technique. Step 1) the needle is inserted into the skin at a 45 degree angle, and negative pressure is applied until blood return is visualized inside the syringe. Step 2) the syringe is removed and guide wire is inserted. Step 3) once guidewire has been advanced to a desired length, the needle is withdrawn. Step 4) Insertion site is enlarged using a scalpel as necessary for large catheters. Step 5) while holding the guidewire, the catheter is advanced into the vein. Step 6) the guidewire is withdrawn and blood return is sought after to verify catheter placement.

FIG. 3 is a schematic of an exemplary system including a detection unit, disposable catheter unit, and wires providing electrical connection.

FIG. 4 is a block diagram of an example system for tracking and indicating a position of a needle.

FIGS. 5A and 5B show an example wrist-mounted interface for indicating a position of a needle.

FIGS. 6A and 6B show an example catheter-mounted interface for indicating a position of a needle.

FIGS. 7A-7C show an example electrode-mounted interface for indicating a position of a needle.

FIG. 8 shows example indicator patterns for indicating different positions of a needle to a user.

FIG. 9 is an example method for determining and presenting an indication of a position of a needle.

FIG. 10 is a diagram schematically depicting a computing environment suitable for implementation of disclosed technologies.

FIG. 11 shows an example circuit diagram of a detection circuit.

FIG. 12 shows an example internal circuit diagram of a timer that may be used in a detection circuit.

### DETAILED DESCRIPTION

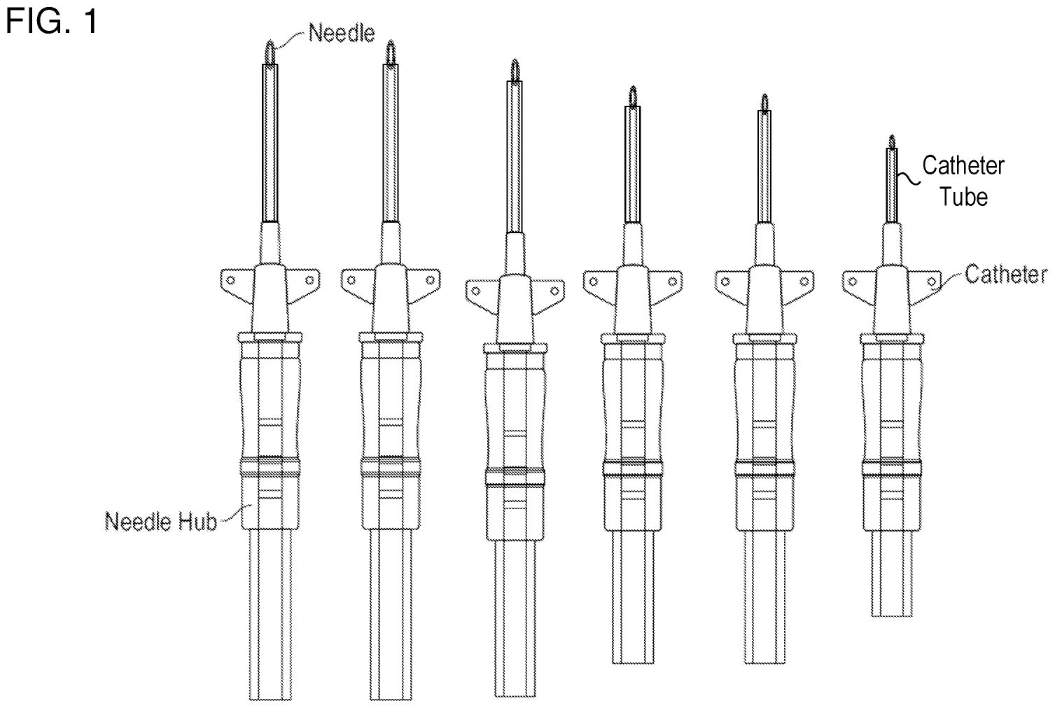

A conventional pIV (see FIG. 1) is placed by inserting the needle into a vein. As venous blood flows into the needle and enters the clear chamber (the “hub”) this is called the “flash”. The user recognizes the flash as evidence of vein entry then advances the catheter into the vein.

The catheter is slightly larger than the needle diameter and set back from the tip of the needle such that the needle may enter the vein without the catheter being in the vessel thus preventing advancement. The “flash” depends on venous pressure, which must be sufficient to drive blood flow into the clear chamber through the needle. Normal venous pressure is only 2-3 mm Hg but can be lower in patients experiencing shock or dehydration. Visualization of the “flash” can be delayed or prevented by high needle resistance found in smaller gauge IVs such as those employed in pediatrics. As a result, many users continue to advance the needle until passing through the vein before visualizing a flash resulting in a “blown vein.”

Once the needle is inserted into the vein the catheter is advanced over the needed and into the vein. Though tapered, the catheter can be larger than the hole in the vein, which can cause some resistance to entry, at times injuring or tearing the vein, which is an additional complication that may occur. When the catheter is sufficiently inserted, the needle can then be removed and tubing is connected to the catheter for administration of medications and fluids.

As discussed, the inability to advance the catheter after an initial blood flash attributed to the needle not yet being in the anterior portion of the vein or the needle passing through the posterior portion of the vein (“blown vein”), glancing or tearing of the vein due to resistance to catheter advancement, and medication leaking into surrounding tissue as a result of a shallow position within the vein resulting in subsequent dislodgement (infiltration), represent the major failures in the present technology that result in failed first attempts.

Technologies intended to improve pIV placement include ultrasound guidance and integrated Seldinger devices (e.g., devices marketed under the name AccuCath). However, present ultrasound technology, when used in cross section often does not permit the user to recognize vessel entry so blood return is still sought after. Ultrasound use in longitudinal section (section obtained by slicing in any plane parallel to the vertical axis) is extremely challenging for small veins even for seasoned clinicians. Furthermore, ultrasound does nothing to assist in catheter advancement. As a result, even with extensive training ultrasound employed in “difficult” patients has been shown to have a first attempt pIV failure rate of 31% and 29% in adults and 58% in pediatrics. In addition, additional time (2-4 min) is required for use of ultrasound as it requires an additional device and added sterile precautions. In a center using ultrasound routinely, nurses still regard 22% of patients to be “difficult” to obtain vascular access.

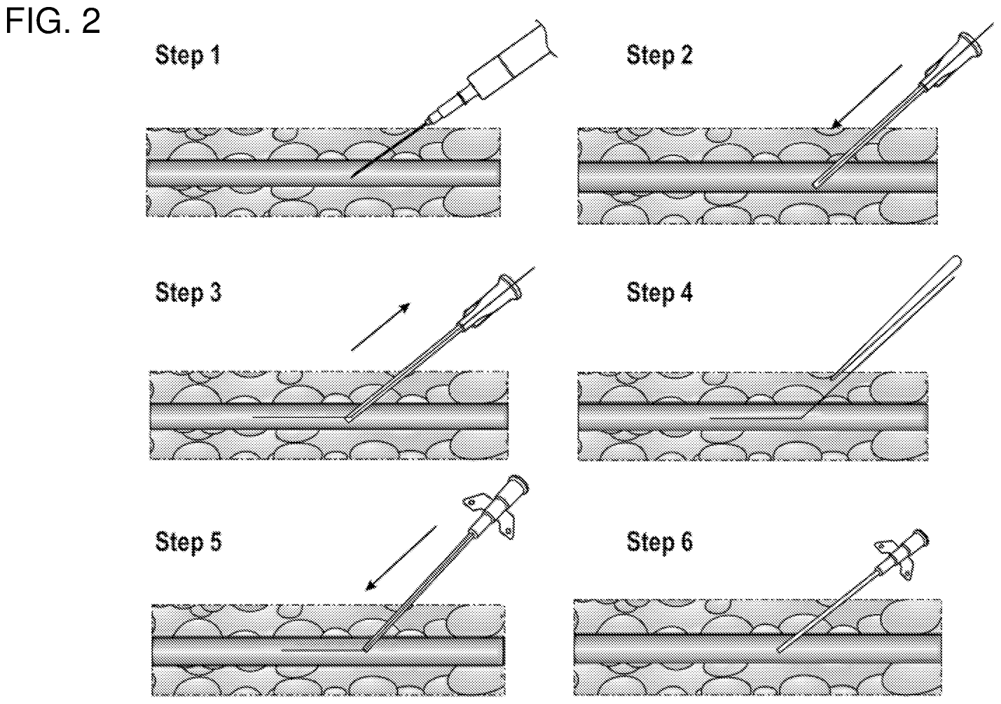

Most often during central venous or arterial catheter placement, the Seldinger technique is employed by the end users in order to obtain access to veins. The Seldinger technique (see FIG. 2) involves six individual steps which are described below.

Step 1) the needle is inserted into the skin at a 45° angle, and negative pressure is applied until blood return is visualized inside the syringe.

Step 2) the syringe is removed and guidewire is inserted into the vein through the needle.

Step 3) once guidewire has been advanced to a desired length, the needle is withdrawn while holding the guidewire.

Step 4) Using a scalpel and dilator, the insertion site is enlarged as necessary for large catheters.

Step 5) the catheter is advanced into the artery/vein using the guide wire.

Step 6) the guidewire is withdrawn, and blood return is sought after to verify catheter placement.

The Seldinger technique can require a complete sterile set up and considerable training. A simple integrated Seldinger system exists that integrates the guidewire into the plastic chamber behind the needle (where blood return is visualized). Such “AccuCath” systems suffer from the shortcoming that the integrated guidewire creates and additional obstruction to blood flow. In the setting of low venous blood pressure (2-3 mm Hg) this prevents the user from visualizing the blood flash. The disclosed technology obviates the need to visualize the flash confirming vein entry by change in electrical resistance and notifying the user through a light, sound, or vibration (selected by the user). Thus the guide-wire in the Seldinger technique can be adopted to gain its advantages in catheter placement while overcoming the disadvantage created by obstructing blood return.

In the field, paramedics have turned to intraosseous (i.e. needle into bone; IO) access due to randomized trials demonstrating 91% first attempt success with IO placement after cardiac arrest vs. 43% first-attempt success rate for pIV. As a result, in emergency situations the present recommendations have shifted to favor IO placement rather than standard pIV. But IO access is not a primary alternative to pIV's due to the increased discomfort experienced by the patient.

In some situations, vein finders use near infrared light (e.g., 628 nm) to visualize veins. However, these devices merely identify where a vein is—they do not necessarily help with placement of a device in the vein. As a result, near-infrared vein finder devices have been found to not improve cannulation, thus failing to address the root causes of pIV placement failure.

In some applications, the disclosed technology can be applied to the “difficult” patient when it comes to establishing pIV vascular access. Roughly 25% of all patients fit this category where multiple attempts are required. Fortunately, these patients can be prospectively identified by skilled nurses. If one uses the conservative (because it only reflects adults and excludes more challenging pediatrics) 2.2 pIV attempts per patient, then the 1.2 billion pIV systems purchased worldwide annually reflect 545 million patients, 136 million of whom fall into the “difficult” category (i.e., expected to require multiple attempts).

The disclosed technology can improve patient care and outcomes as well as healthcare system performance by accelerating successful pIV placement, which can result in one or more of the following benefits/advantages:
1. Reduced patient pain perception, bruising and hence improved satisfaction.
2. Reduced adverse events including infiltrations, phlebitis, infections and bruising all of which are associated with multiple pIV attempts. Though uncommon, extravasation of caustic agents can result in significant litigation.
3. More rapid delivery of potentially life-saving therapies such as blood, fluid or antibiotics.
4. Reduction in cost to healthcare systems that are not reimbursed for failed attempts. This cost savings would be realized for example by the ability to reduce staffing on the IV team from 4 to 3 nurses per shift.

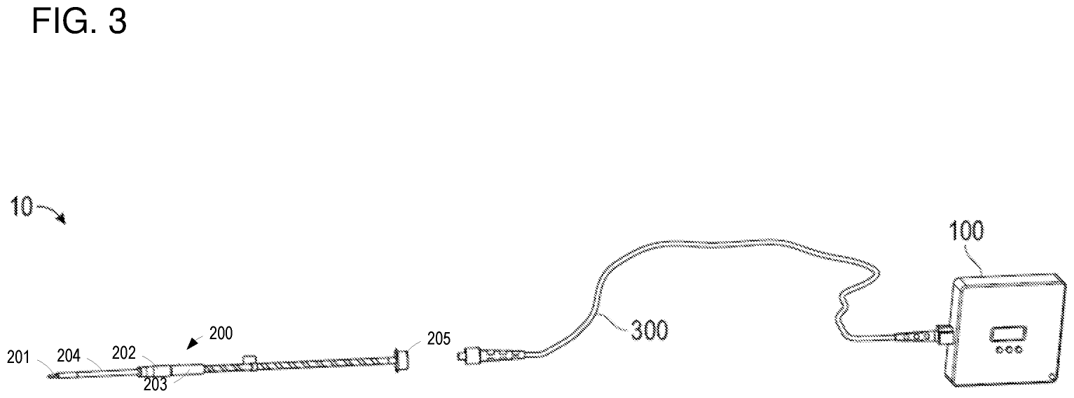

FIG. 3 shows an exemplary catheter insertion system 10 that comprises a detection unit 100, a disposable catheter unit 200, and wires 300 providing the electrical connection to the detection unit. Several exemplary system combinations are presented in this application for the detection unit and the disposable catheter unit, both of which are described below in greater detail. The catheter unit 200 can include a needle 201, a removable catheter 202, needle housing 203, a catheter tube 204, and an electrical connector 205. The electrical connector 205 attaches to a mating connector from a non-disposable cable 300, as also shown in FIG. 3. In some examples, the needle 201 may include a guidewire threaded therethrough, examples of which are described in more detail in U.S. Application Publication No. 2020/0206471, incorporated by reference herein in its entirety.

The catheter insertion system 10 measures the electrical resistance of materials that the needle contacts as it is inserted. The resistance values can be used to indicate progress of the needle through subcutaneous tissue and into the vein and in contact with blood. This information can be used through various algorithms and hardware to allow the IV nurse to successfully place the catheter on the first attempt.

The tissue or fluid generally constitutes an electrical impedance that may be simplified to be considered a resistor that can be measured by different techniques. Some embodiments of the detection circuit (described below) contain an oscillator whose frequency of oscillation depends on the quantities of connected resistor and capacitor components. In some embodiments, the electrical resistance of the tissue or fluid between the needle and guide-wire make up a key resistor component in the circuit. Different resistances (e.g., fatty tissue under the skin vs. blood inside the vessel) cause the frequency of oscillation to change. By measuring this frequency, the type of tissue in contact with the needle, and thus the location of the needle can be determined.

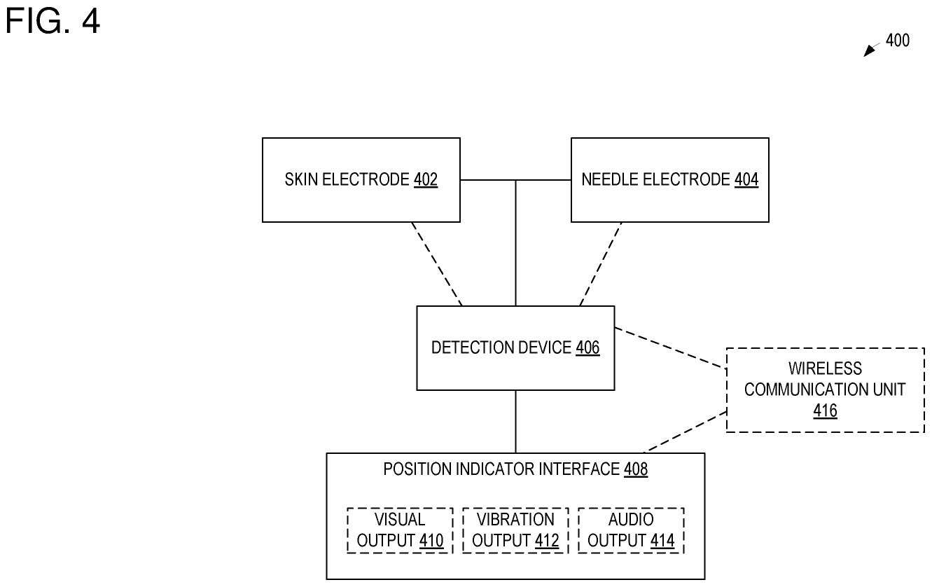

FIG. 4 is a block diagram of an example system 400 for detecting a position of a needle (e.g., as the needle is progressed through subcutaneous tissue to reach an interior of a vein) and providing output indicating the position of the needle. As a needle is inserted into the body, the tip of the needle will encounter various tissue types on the path to its intended destination. For example, during pIV catheter placement the needle passes through the outer skin and subcutaneous tissues, then the vessel wall, then the blood inside the vessel (and possibly the backside wall and other tissues if the needle were to exit the vessel). Herein the term “position” or phrase “position of the needle” refers to the inference of where the needle is located in the body based on the measurement of the type of tissue the needle is encountering. For example, a determined position of the needle may refer to an inferred location of the needle relative to one or more of the various tissue types described above (e.g., outside of the body, within subcutaneous tissue outside of the vessel wall, within the vessel wall, within blood inside the vessel, within muscle, within a dermal layer, etc.). The system 400 includes at least a pair of electrodes, including a skin electrode 402 (also referred to as a “dermal electrode”) and a needle electrode 404. For example, the skin electrode may be adhered to or otherwise disposed on a surface (e.g., an outer surface) of a skin of a patient (e.g., with a conductive gel between the skin electrode and the patient's skin). The needle electrode 404 may be disposed in any suitable location of a needle of a catheter (examples of needles are shown and described with respect to FIGS. 1-3), such as on a body of the needle, a tip of the needle, a guidewire within the needle, etc. An advantage is that by using the skin electrode, fabrication of the needle/catheter is simpler as only one electrode is needed on the needle, and it can be the needle itself. Otherwise, the system would utilize two electrodes to be placed on the needle, which requires multiple layers of insulation and metals and/or patterning, etc. In some examples, there may be a benefit of using a guidewire in the needle as an electrode. If the skin electrode is used, then the guidewire and needle can be electrically connected, which again is simpler to fabricate. It is to be understood that more than two electrodes may be used, for example, a plurality of skin electrodes and/or a plurality of needle- or guidewire-based electrodes may be used in some examples. In additional or alternative examples, in addition to one or more skin electrodes and/or one or more needle electrodes (which may include one or more guidewire electrodes), one or more further electrodes may be utilized. For example, implanted stimulation electrodes, such as spinal cord implanted electrodes, may be utilized in combination with the electrodes described herein to determine a position of a needle. Furthermore, the skin electrode may include microneedles to improve electrical connection with the skin tissue.

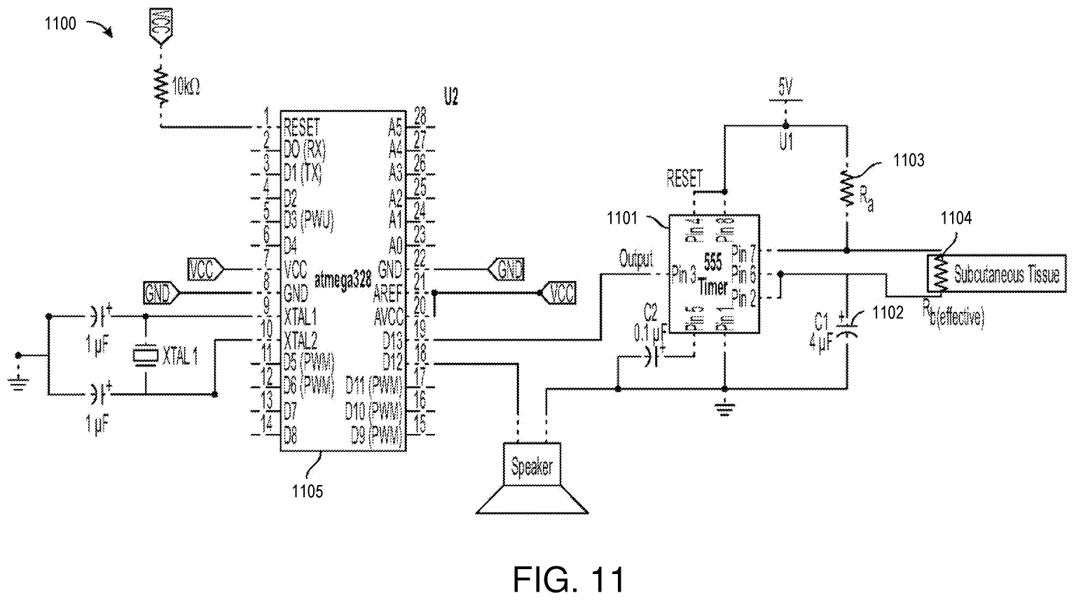

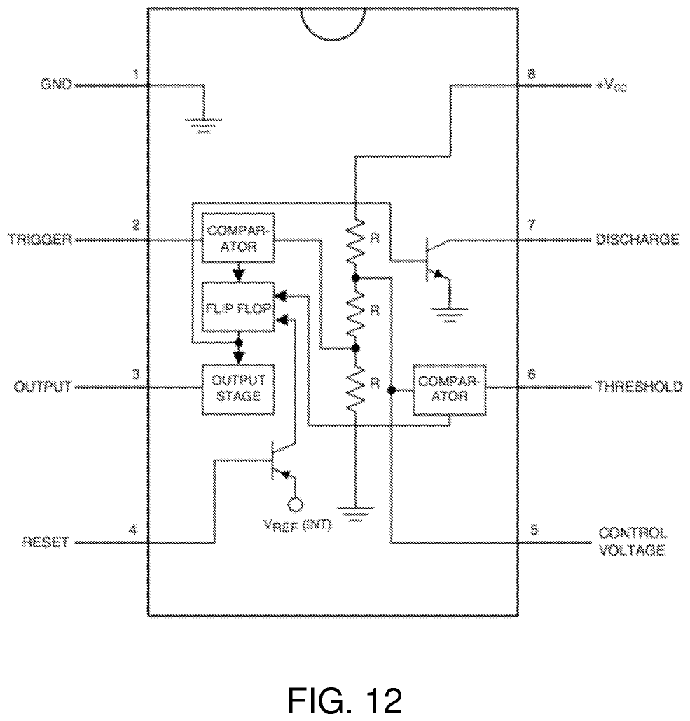

A detection device 406 may include a computing system and/or circuitry configured to receive signals from the skin electrode 402 and the needle electrode 404 and determine a position of the needle and/or determine a progress of the needle through tissue (e.g., to measure resistance between the skin electrode and the needle electrode to determine an anatomical space within which an end portion of the catheter unit is located within the patient based on the measured resistance). Examples of circuitry that may be used to process the signals from the electrodes to determine the position and/or progression of the needle are described below in FIGS. 11-12 and in U.S. Application Publication No. 2020/0206471, which is incorporated by reference herein in its entirety. For example, tissue and/or fluid resistance may be analyzed as a resistor that can be measured by different techniques. In one example, the detection device 406 includes a detection circuit, which includes an oscillator whose frequency of oscillation depends on the quantities of connected resistor and capacitor components. In such examples, the tissue and/or fluid resistance between the skin electrode and the needle electrode make up a key resistor component in the circuit. Different resistances (e.g., fatty tissue under the skin vs. blood or muscle tissue) cause the frequency of oscillation to change. By measuring this frequency, the type of tissue in contact with the needle, and thus the location of the needle can be determined.

Turning briefly to FIGS. 11 and 12, example circuits for a detection device are shown. FIG. 11 shows an example circuit diagram of a detection circuit, while FIG. 12 shows an example internal circuit diagram of a timer that may be used in a detection circuit. FIG. 11 displays an example of a circuit diagram (as described above) for a detection unit 1100, although other types of timing circuits may be used. The circuit includes a timer chip 1101 (for example a 555 chip) a capacitor of pre-determined value 1102 (for example a 4.7 μF capacitor), resistors Rₐ 1103 (pre-determined, for example 675Ω), R_{effective} 1104 the unknown resistance of the tissue and a microcontroller 1105. As shown, two electrodes are connected to the circuit through extension wires and the tissue/fluid resistance becomes the effective resistance R_{effective}. Although only subcutaneous tissue is shown in the figure, it should be noted that the cannula encounters other materials such as blood and muscle during use. The 555 is a timer chip that is used to generate time delays or oscillation. It has two modes of operation, mono stable (time delay), and astable (oscillator). A preferred use in this system is in astable mode.

The operation of the 555 timer chip (as well as other example timer circuits) is described here to clarify how it is used to measure tissue/fluid resistance in the disclosed system. FIG. 12 displays the internal circuitry of the 555 timer. The internal circuit of the timer consists of three 5KΩ resistors, two comparators that compare two input voltages (labeled V+ and V−), a flip flop, an output stage and two transistors. In astable mode, a voltage (Vcc) is provided across the resistors Rₐ and R_{effective} (the unknown tissue/fluid resistance) 1104, which in turn starts charging the capacitor 1102. Once the capacitor reaches some percentage of the supply voltage (for example ⅔) it discharges through the transistor in pin 7 and R_{effective}. Once discharged the capacitor starts re-charging through resistors Rₐ and R_{effective}. While the capacitor is charging, the first comparator connected to pin 2 compares the input voltage from the trigger pin to a reference voltage that is a percentage of Vcc (for example, ⅓). At the same time, the second comparator compares the input voltage from the threshold pin (pin 6) to a reference voltage (for example, ⅔ Vcc) from the voltage divider. When the input voltage (V+) is higher than the reference voltage (V−) the comparator outputs a logic 1 or if V− is higher than V+ then the comparator outputs a logic 0.

The outputs from the two comparators are connected to the flip flop which produces either a logic 1 or a logic 0 signal based on the state of the inputs. Next, the output signal from the flip flop travels to the output stage. When the output stage receives a logic input of 0 from the flip flop it outputs a digital high voltage at that time. Subsequently when a logic input of 1 is received by the output stage, pin 3 is connected to ground, and the transistor in pin 7 is opened allowing the capacitor to discharge. This process continuously repeats while the timer is operating in astable mode producing a clocking signal (oscillating binary output in the form of a rectangular wave) outputted via pin 3 whose signal is sent to a microcontroller (e.g., ATmega328p). The frequency of the rectangular wave is dependent on the relative values of the resistors 1103 and 1104 and the capacitor 1102 and in this scenario is used to determine the resistance or change in resistance of the unknown tissue 1104. Other component values could be determined using related methods. While use of the 555 timer chips is one method for relating resistance to oscillation frequency, it is not the only method that can be used. Any suitable method that uses a time-constant of a resistor-capacitor or resistor-inductor circuit to create a dynamic response or an oscillating signal can be used as well to relate the time characteristics of the signal to the unknown resistance, capacitance, and/or inductance.

The microcontroller 1105 is responsible for measuring the frequency of the signal produced by the timer chip (pin 3). There are several options for conveying a detected change to the end user. One option is based on the absolute value of the measured frequency (or resistance) and the other is based on a change in measured frequency (or resistance).

When using the absolute value method, a threshold can be set (e.g. frequency<100 Hz for fatty tissue) the end user can be alerted to contact with muscle or blood through output interfaces if the measured frequency value is greater than the specified cutoff. (Note that in the circuit 1100 described here, signal oscillation frequency is inversely dependent on resistance 1104, so as resistance decreases, for example when the electrodes pass from fatty tissue to blood, the signal frequency increases. Other circuits could be configured so as to produce a proportional relationship between frequency and resistance. In addition, methods in which the duty cycle is measured as related to an unknown resistance, capacitance, or inductance are also viable approaches.) Setting an absolute threshold is ideal when a large separation exists between the two quantities being compared. Conversely, the absolute value method presents a problem if the two quantities being compared (e.g. blood vs. muscle) do not have a significant separation between them.

An alternative is to look for a change in baseline (or nominal or initial) frequency due to a change in resistance. This would be accomplished by setting the initial value when the cannula (electrodes) first enters the tissue, for example when the measured resistance changes from air (open circuit) to skin and/or fatty tissue. The frequency observed when the electrodes are in fatty tissue can be set as the baseline and for example can be stored in memory. As the cannula is advanced the user can be alerted to the change when the initial recorded frequency value rises by a certain amount (e.g. 25% increase). The algorithm within the microcontroller could monitor absolute value compared to a threshold, percentage change compared to a baseline, a combination of these changes, or other methods are possible.

The relationship between the rectangular wave frequency and the unknown resistance value (R_{effective}) of the tissue/fluid is described by Equation 1 below. Solving Equation 1 for R_{effective} as shown by Equation 2 below provides an expression for the unknown resistance as a function of the measured frequency. It is not necessary to convert the measured frequency values to resistance. This is possible because subcutaneous tissue and blood exhibit distinctive frequencies when their resistance is measured in this way that allow for differentiation between the two quantities and detection of vessel entry. The nominal output frequency of the system is controlled by selecting the values of the resistor Rₐ and capacitor C. Choosing a large capacitor value increases the cycle time of the system, which in turn reduces output frequency; and increasing Rₐ increases the high time (the amount of time spent at the top of the rectangular wave) while leaving the low time (the amount of time spent at the bottom of the rectangular wave) unaffected. The respective values of C (4.7 μF) and Rₐ (675Ω) are shown as examples that produce reasonable separation between subcutaneous tissue and blood, but many other values are feasible.

$$f = \frac{1}{T} = \frac{1.44}{( {R}_{a} + 2 {R}_{effective} ) C} \qquad ( 1 )$$

$${R}_{effective} = \frac{1}{2} ( \frac{1.44}{fC} - {R}_{a} ) \qquad ( 2 )$$

Another alternative to using a timer circuit or another oscillating circuit for measuring the unknown tissue/fluid resistance is to utilize a Wheatstone bridge and alternating current (AC). Unlike DC bridges, where the resistance can be directly measured, AC bridges measure the impedance. An AC bridge may be used instead of DC in order to negate the effect of polarization. Applying a direct current to a liquid solution causes an accumulation of ions near the surface of the electrodes which leads to the polarization of the measurement electrodes and thus erroneous results. Applying alternating current forces the ions to continuously migrate from one electrode to the other thus effectively negating the effect of polarization. In examples where more than one electrode is utilized, signals from all of the electrodes may be processed by the detection unit to determine the position of the needle to a greater degree of accuracy compared to examples in which two electrodes are used.

Returning to FIG. 4, the detection device 406 may include a battery to power the device, timer circuit (or alternative oscillatory component or circuit), microcontroller (frequency measurement and interface control), and/or other components to process the incoming signals from the electrodes and generate output indicative of a position and/or progress of the needle through tissue. In some examples, some or all of the components of the detection device may be mounted on a printed circuit board and/or included in a housing. In some examples, the housing may be included in, integrated with, and/or mounted on a needle assembly (e.g., a disposable catheter assembly as shown in FIG. 3) that includes the needle. In other examples, the housing may be separated from the needle and attached to the electrodes via respective wires. For example, the detection device 406 and/or components of the detection device 406 may be included in a handheld or other computing device, such as a wristwatch (e.g., a smartwatch or other wrist-worn computing device), a mobile phone (e.g., a smartphone), a laptop, a desktop computing device or other monitoring device, etc.

A position indicator interface 408 may receive data (e.g., signals, including digital and/or analog data) indicating a position of the needle and/or a progression of the needle through tissue from the detection device 406. Based on the received data, the position indicator interface 408 may generate output indicating the position of the needle and/or the progression of the needle through tissue. For example, the output may include visual output 410, vibration output 412, and/or audio output 414. Examples of visual output 410 may include a graphical user interface that includes a display (e.g., a liquid crystal display or other alpha numeric or graphical display) and/or one or more light outputs (e.g., light emitting diode(s) [LED] or LED array(s)) configured to output a visual representation of the indication of the position and/or progression of the needle. Examples of vibration output 412 may include a vibratory, haptic, and/or other tactile interface configured to output tactile feedback indicating the position and/or progression of the needle. Examples of audio output 414 include a speaker and/or other audio output device configured to output audible feedback indicating the position and/or progression of the needle.

In some examples, the system 400 may include a wireless communication unit 416, which may include a radio component (e.g., radiofrequency [RF], near-field communication [NFC], Bluetooth, WiFi, or other suitable component) that enables the system to communicate wirelessly to a remote device, such as a mobile device (e.g., a cell phone, smartwatch, or other mobile receiving unit) or a network or a computer such that the information (measured frequencies and/or electrical impedance values, indications of position and/or progression of the needle, and/or generated output indicating the position and/or progression of the needle) can be transferred to such devices, computers, and/or networks. Software applications can execute on the devices (e.g., an app on a mobile phone or computer or software on a server) that can receive, analyze, and/or store the data (for example in a database in a server). In such output (e.g., lights, sounds, vibrations, etc.) can be presented on the mobile device or on some other device connected to a computer in addition to or in place of the output interface(s) 408 of the system 400.

A software application on a mobile device or computer can be configured to enable the hardware (electrode system, sensing/detection device, or a combination) to operate the same or differently for medical procedures other than pIV placement. For example, placement of pleural, pericardial or peritoneal catheters. In such a scenario, for example in which a cell phone is wirelessly connected to the detection unit, the user could select in the app what procedure is to be executed, and information could be transferred from the phone to the detection unit to establish operating methods in the microcontroller. For example, one or more parameters could be passed to the microcontroller to indicate that pIV is the procedure of interest, so associated frequency or impedance values can be measured or passed back to the mobile device or computer or network to be analyzed, stored (for example in a database on the network or in the mobile device or computer) or to be used to alert the user. The information transferred from the detection unit could be measurement of frequency or resistance at specific times (e.g., periodically) or based on events (e.g., changes in frequency or alerts that a frequency threshold has been crossed), or it could be alerts that certain events have occurred. Alternatively, data from the detection unit could be streamed in real time to the mobile device or computer or network so that it may be analyzed in real time to be used immediately by the user or be stored for future use.

In some examples, one or more of the components of system 400 may be included in, integrated with, and/or permanently or separably connected to the catheter system itself. The component(s) may be miniaturized (including the components of FIG. 4) onto a printed circuit board or as a system-on-a-chip such that it can be included as an integral part of the catheter system (200) of FIG. 3 or other suitable catheter system, thereby eliminating the need for a separate detection unit shown in FIG. 3. In such case there may be a radio included for wireless communication with a detection unit, or a mobile device as described above, or circuit(s) could be connected directly to the mobile device or computer through wires. When wired directly, certain functions can be carried out by the mobile device or computer thereby allowing elimination of that related component from the detection circuit, for example eliminating the microcontroller in the detection circuit (and carrying out the analysis and control on the mobile device), or eliminating the battery (whereby the detection circuit is powered from the mobile device through wires), or eliminating the timer circuit (in which case timing or oscillator generator or similar function is executed on the mobile device or computer), or eliminating the output interface (and using the user interfaces on the mobile device to relay information and alerts to the user), or eliminating combinations of these components.

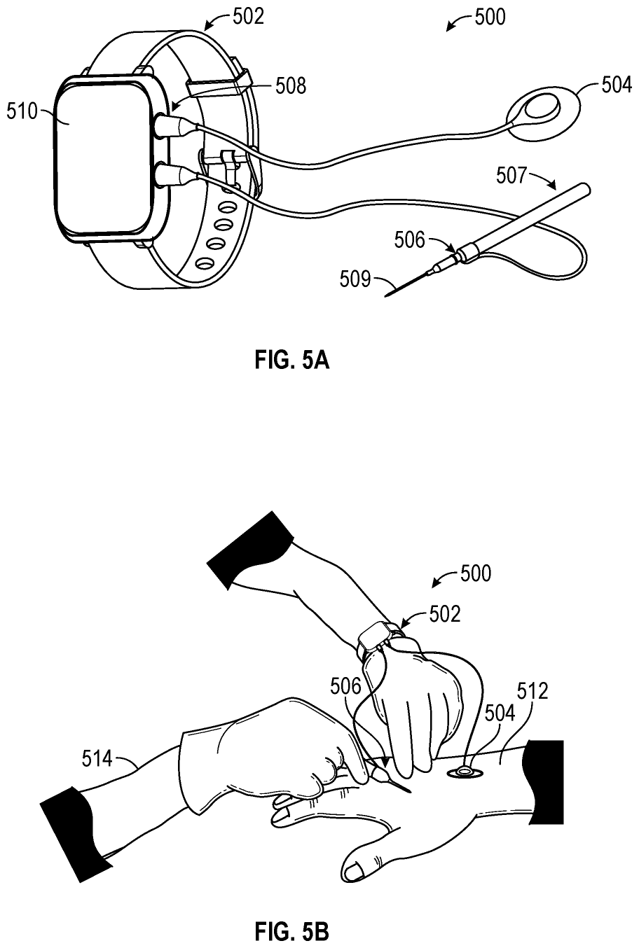

FIGS. 5A and 5B show an example configuration of a needle position feedback system 500 that utilizes a smartwatch or similar wrist-worn user interface and computing device to provide feedback to a user of a position and/or progression of a needle through tissue. The system 500 includes a smartwatch 502, serving as a detection device and in some cases position indicator interface (e.g., an example of detection device 406 of FIG. 4 and position indicator interface 408 in FIG. 4), which is connected (e.g., via wires) to a skin electrode 504 and a needle electrode 506 of a catheter system 507. The electrodes may connect to the smartwatch 502 via a connection interface 508 that accepts connectors of wires carrying respective signals from the electrodes 504 and 506. The smartwatch 502 may include a display 510 configured to provide a graphical user interface, which may provide visual feedback of a position and/or progress of a needle 509 of the catheter system 507 in some examples, as described in more detail above with respect to FIG. 4. In additional or alternative examples, the smartwatch 502 may include a haptic and/or vibratory output system and/or a speaker system configured to provide tactile and/or audible (respectively) feedback of a position and/or progress of the needle 509, as described in more detail above with respect to FIG. 4. As shown in FIG. 5B, the skin electrode 504 may be positioned on the skin of a patient 512 and a position of the needle in passing through tissue of the patient 512 may be fed back to a clinician or other healthcare professional 514 via the smartwatch 502 based on signals received at the smartwatch via the electrodes 504 and 506 as described above.

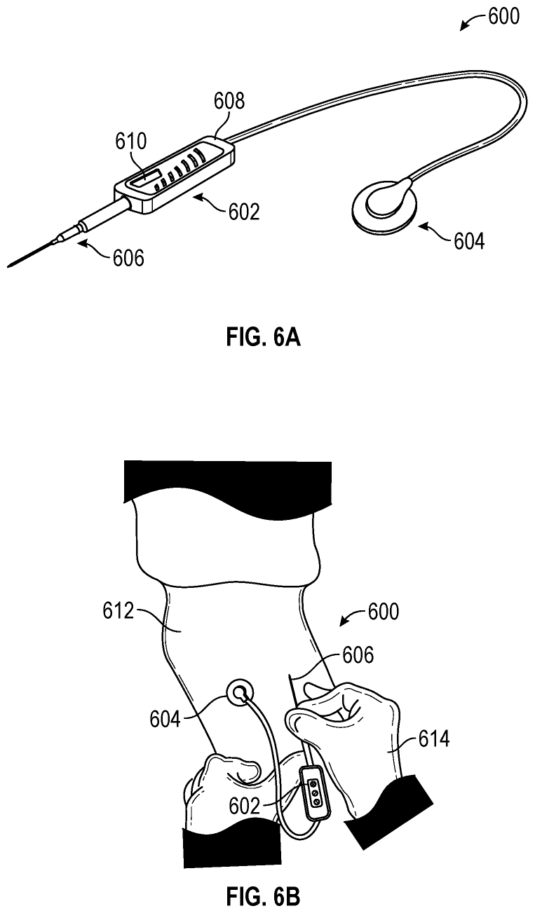

FIGS. 6A and 6B show an example configuration of a needle position feedback system 600 that utilizes a catheter-mounted indicator display to provide feedback to a user of a position and/or progression of a needle through tissue. The system 600 includes a sensing unit 602, serving as a detection device (e.g., an example of detection device 406 of FIG. 4), which is connected (e.g., via one or more wires) to a skin electrode 604 and a needle electrode of needle 606 (e.g., a sensing tip of the needle 606). The sensing unit 602 may be mounted on a catheter system that includes the needle 606. The electrode 604 may connect to the sensing unit 602 via a connection interface 608 that accepts connectors of wires carrying signals from the electrode 604. The electrode of the needle 606 may connect to the sensing unit 602 in any suitable manner, such as through a connection associated with mounting components that allow the sensing unit to snap onto the needle and/or via one or more wires in a similar manner as shown for the connection with the electrode 604. The sensing unit 602 may include a display 610 configured to provide a graphical user interface, which may provide visual feedback of a position and/or progress of the needle 606 of the catheter system in some examples, as described in more detail above with respect to FIG. 4. In additional or alternative examples, the sensing unit 602 may include a haptic and/or vibratory output system and/or a speaker system configured to provide tactile and/or audible (respectively) feedback of a position and/or progress of the needle 606, as described in more detail above with respect to FIG. 4. As shown in FIG. 6B, the skin electrode 604 may be positioned on the skin of a patient 612 and a position of the needle 606 through tissue of the patient 612 may be fed back to a clinician or other healthcare professional 614 via the sensing unit 602 based on signals received at the sensing unit via the skin electrode 604 and the electrode on the needle 606 as described above.

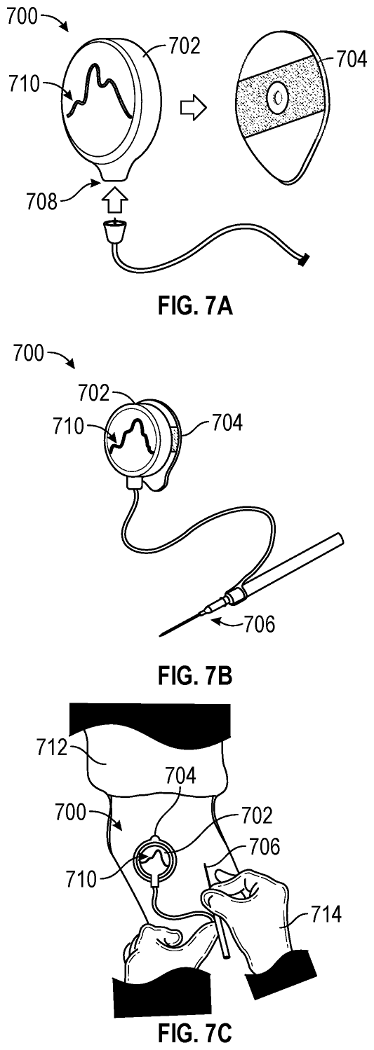

FIGS. 7A-7C show an example configuration of a needle position feedback system 700 that utilizes an electrode-mounted indicator display to provide feedback to a user of a position and/or progression of a needle through tissue. The system 700 includes a sensing unit 702, serving as a detection device (e.g., an example of detection device 406 of FIG. 4), which is connected (e.g., via on one or more wires) to a needle electrode of needle 706 (e.g., a sensing tip of the needle, where the needle may be disposable and the sensing unit may be reusable in some examples). The sensing unit 702 may be mounted on a skin electrode 704 (e.g., an electrode included and/or embedded on a patch or other component that may adhere or otherwise be placed onto a patient's skin, such as a stick-on patch). The electrode of needle 706 may connect to the sensing unit 702 via a connection interface 708 that accepts connectors of wires carrying signals from the electrode of needle 706. The skin electrode 704 may connect to the sensing unit 702 in any suitable manner, such as through a connection associated with mounting components that allow the sensing unit to snap onto the skin electrode 704 and/or via one or more wires in a similar manner as shown for the connection with the electrode of the needle 706. The sensing unit 702 may include a display 710 configured to provide a graphical user interface, which may provide visual feedback of a position and/or progress of the needle 706 of the catheter system in some examples, as described in more detail above with respect to FIG. 4. In additional or alternative examples, the sensing unit 702 may include a haptic and/or vibratory output system and/or a speaker system configured to provide tactile and/or audible (respectively) feedback of a position and/or progress of the needle 706, as described in more detail above with respect to FIG. 4. As shown in FIG. 7C, the skin electrode 704 may be positioned on the skin of a patient 712 and a position of the needle 706 through tissue of the patient 712 may be fed back to a clinician or other healthcare professional 714 via the sensing unit 702 based on signals received at the sensing unit via the skin electrode 704 and the electrode on the needle 706 as described above.

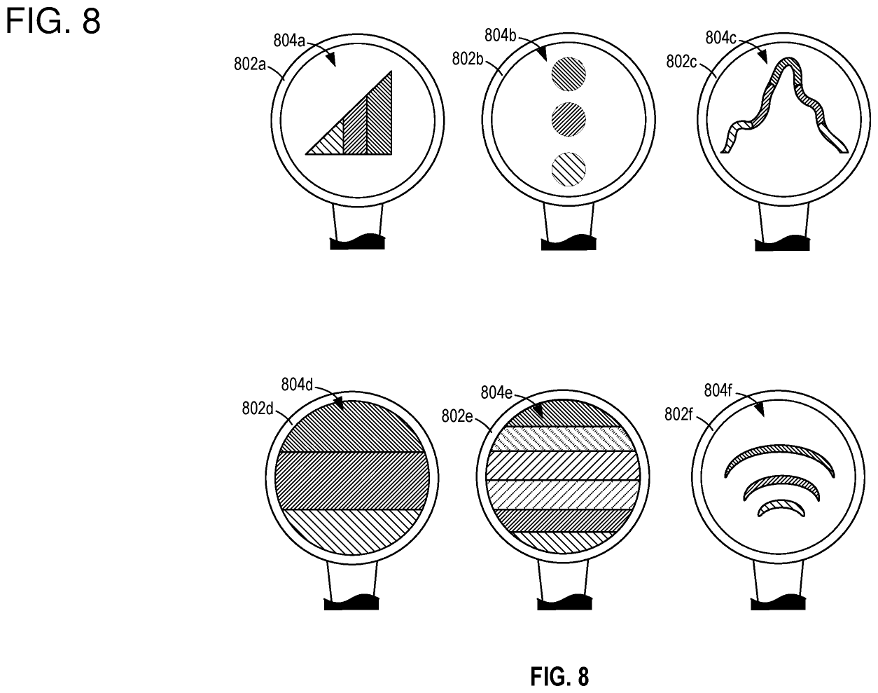

FIG. 8 shows example indicator displays 802a-802f showing example indicator visuals 804a-804f. The example indicator visuals 804a-804f of FIG. 8 may be used (e.g., displayed on an associated display device) in some examples of the smartwatch 502 of FIGS. 5A and 5B, the sensing units 602 and 702 of FIGS. 6A-7C, and/or any of the above-described needle position/progress indicators. In each of the visuals of FIG. 8, different colors (e.g., red, orange, green and/or shades therebetween, which are represented by different diagonal fill patterns in the example visuals) may be mapped to different tissue types or to different levels of progress of a needle through tissue and/or a closeness of the needle to a target position (e.g., within a vein and/or touching blood). For example, displaying and/or filling a visual to a red portion of the example visuals may indicate that the needle is in a farthest position grouping (e.g., outside the body), displaying and/or filling a visual to an orange/yellow portion of the example visuals may indicate that the needle is progressing further toward a target position (e.g., contacting an outer dermal layer of a patient, within subcutaneous tissue of the patient but outside of and/or touching an outer surface of a vein, etc.), and displaying and/or filling a visual to a green portion of the example visuals may indicate that the needle has reached a target position (e.g., within a vein in contact with blood, etc.). In other examples an opposite mapping to that described above may be used (e.g., green may indicate that the needle is at a farthest mapped position from the target such as outside of the patient's skin, orange/yellow may indicate that the needle is progressing through tissue, and red may indicate that the needle has reached a target position). The representative colors and shapes of indicators shown in FIG. 8 are exemplary in nature, and other shapes, patterns, colors, and/or combinations thereof may be mapped to different relative positions of the needle to a target position. In additional or alternative examples, different indicators may be used to differentiate between an electrode-based detection of the needle contacting muscle versus fat. In some examples, the indicators may indicate the position of the needle as being in one of a set number of basic positions (e.g., out in air, in skin, and in blood) based on conductivity measured using the electrodes as described above. In other examples, graduated indicators may be used to indicate one or more levels within the above-described basic position groupings.

As described above, in some examples, a position indicator system may include a wireless communication component that allows for the transmission of data to remote devices. Accordingly, in some examples, one or more of the above described indicator systems may be combined with one another and/or with an additional user interface device to provide the same feedback in multiple devices and/or to provide different types of feedback on different devices. For example, one type of visual indication (e.g., one of the visuals shown in FIG. 8) may be provided on a sensing unit mounted on a skin electrode (e.g., as described in FIGS. 7A-7C) and the sensing unit may send a signal to a smartphone or a smartwatch indicating a position of the needle to cause the smartphone or smartwatch to provide the same or a different type of visual representation of the position and/or to provide a different type of position feedback (e.g., haptic, audio, etc.).

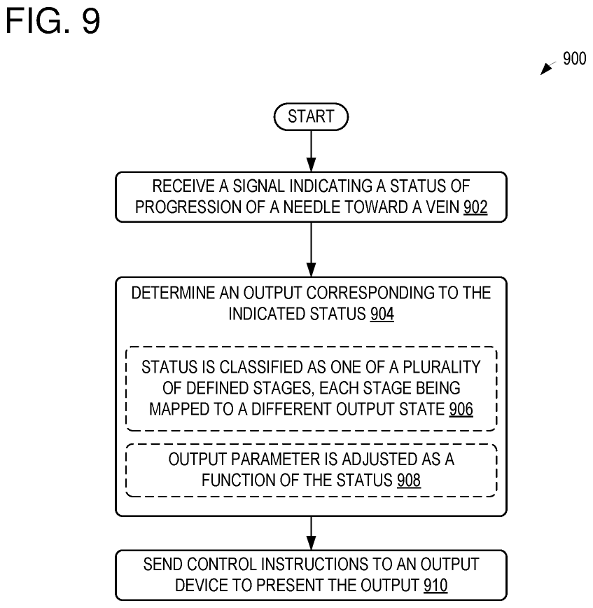

FIG. 9 is an example method 900 of providing an indication of a position and/or progress of a needle toward a target position, such as a vein of a patient. For example, method 900 may be performed by one or more of the example sensing units and/or detection units or systems associated therewith as described above with respect to FIGS. 4-8. At 902, the method includes receiving a signal indicating a status of progression of a needle toward a vein.

At 904, the method includes determining an output corresponding to the indicated status associated with the signal received at 902. For example, as indicated at 906, the status may be classified as one of a plurality of defined stages, each stage being mapped to a different output state. As indicated at 908, an output parameter may be adjusted as a function of the status. For example, the output parameter may include a parameter of an output system, such as a graphical user interface (e.g., for visual indicators), an audio interface (e.g., for audible indicators), a haptic interface (e.g., for haptic indicators), etc. As a more detailed example, the output parameter may include a volume or frequency of an audible output (e.g., for audible indicators), an intensity or pattern of a haptic output (e.g., for haptic indicators), or a color or pattern of visual output. (e.g., for visual indicators, such as the different colors/visuals shown and described above with respect to FIG. 8). At 910, the method includes sending control instructions to an output device (e.g., one or more of the interfaces described above) to present the output.

In a first example, a catheter insertion system comprises a disposable catheter unit comprising a needle and a deployable catheter disposed over the needle, a detection unit coupled to the catheter unit, the detection unit comprising circuitry electrically coupled to the needle and a skin electrode and configured to measure resistance between the needle and the skin electrode while the catheter unit is inserted or being prepared to be inserted into a patient and configured to determine an anatomical space within which an end portion of the catheter unit is located within the patient based on the measured resistance, and an indicator interface configured to send control instructions to an output device to present an indication of the anatomical space within which the end portion of the catheter unit is located within the patient.

A second example includes the first example, and further includes the catheter insertion system, wherein the circuitry is configured to differentiate whether the end portion of the catheter unit is positioned in blood or in subcutaneous tissue based on the measured resistance.

A third example includes one or both of the first and second example, and further includes the catheter insertion system, wherein the anatomical space is mapped to an output state used to indicate a progress of the needle toward a target location.

A fourth example includes one or more of the first through the third examples, and further includes the catheter insertion system, wherein the target location is a vessel of the patient.

A fifth example includes one or more of the first through the fourth examples, and further includes the catheter insertion system, wherein the output device comprises a display.

A sixth example includes one or more of the first through the fifth examples, and further includes the catheter insertion system, wherein the output device comprises a haptic feedback device.

A seventh example includes one or more of the first through the sixth examples, and further includes the catheter insertion system, wherein the output device comprises a speaker system.

An eighth example includes one or more of the first through the seventh examples, and further includes the catheter insertion system, wherein the detection unit comprises a wrist-worn computing device, and wherein the skin electrode and an electrode on the needle is connected to an interface of the wrist-worn computing device.

A ninth example includes one or more of the first through the eighth examples, and further includes the catheter insertion system, wherein the detection unit is mounted on the skin electrode.

A tenth example includes one or more of the first through the ninth examples, and further includes the catheter insertion system, wherein the detection unit is mounted on the catheter unit.

An eleventh example includes one or more of the first through the tenth examples, and further includes the catheter insertion system, wherein one or more of the display, the haptic feedback device, and/or the speaker is included in the detection unit.

A twelfth example includes one or more of the first through the eleventh examples, and further includes the catheter insertion system, further comprising a wireless communication component configured to communicate additional control instructions to a second output device remote from the detection unit.

In a thirteenth example, a method of indicating a position of a needle of a catheter system comprises receiving a signal indicating a status of a progression of the needle toward a vessel of a patient, determining an output corresponding to the indicated status, and sending control instructions to an output device to present the output.

A fourteenth example includes the thirteenth example, and further includes the method, wherein the status is classified as one of a plurality of defined stages, and wherein each stage is mapped to a different output state.

A fifteenth example includes one or both of the thirteenth and the fourteenth examples, and further includes the method, wherein an output parameter is adjusted as a function of the status.

A sixteenth example includes one or more of the thirteenth through the fifteenth examples, and further includes the method, wherein the signal output by a detection unit based on signals from a skin electrode mounted on a skin of the patient and a needle electrode included in the needle, and wherein the detection unit comprises the output device.

A seventeenth example includes one or more of the thirteenth through the sixteenth examples, and further includes the method, further comprising presenting, via the output device, one or more of a visual indication, a haptic indication, and/or an audible indication of the indicated status.

In an eighteenth example, an indicator system for a catheter insertion system comprises a detection unit comprising circuitry electrically coupled to a needle of a catheter unit and to a skin electrode and configured to measure resistance between the needle and the skin electrode while the catheter unit is inserted or being prepared to be inserted into a patient and configured to determine an anatomical space within which an end portion of the catheter unit is located within the patient based on the measured resistance, and an output device configured to present an indication of the anatomical space within which the end portion of the catheter unit is located within the patient based on a signal generated by the detection unit.

A nineteenth example includes the eighteenth example, and further includes the indicator system, wherein the output device comprises a display, a haptic device, and/or a speaker configured to present the indication.

A twentieth example includes one or both of the eighteenth and nineteenth examples, and further includes the indicator system, wherein the indication indicates a status of a progression of the needle toward a vessel of a patient, and wherein an output parameter is adjusted as a function of the status, the output parameter comprising one or more of a volume or frequency of an audible output, an intensity or pattern of a haptic output, or a color or pattern of visual output.

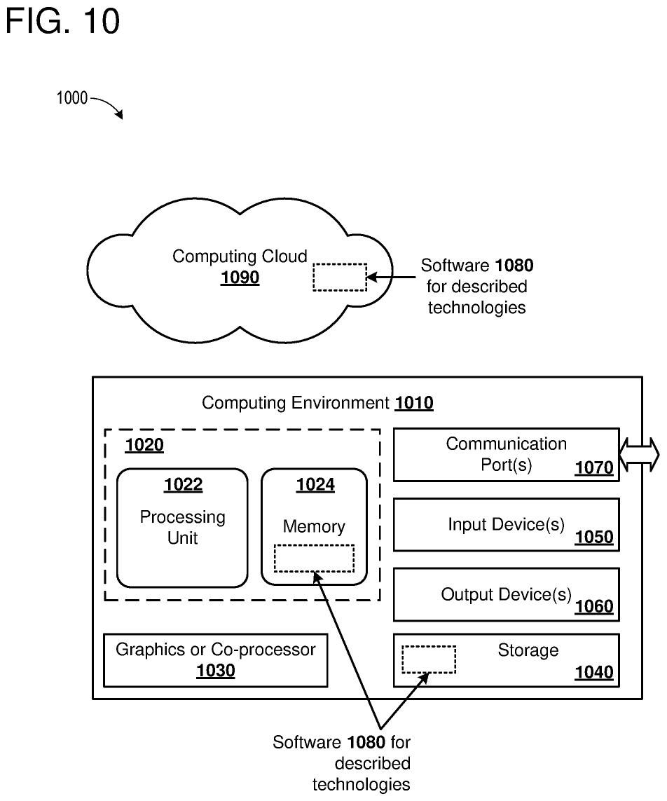

FIG. 10 illustrates a generalized example of a suitable computing system 1000 in which described examples, techniques, and technologies, including scheduling computing operations in an environmental impact-based scheduling interface according to disclosed technologies can be implemented. For example, the computing system 1000 and/or one or more elements of the computing system 1000 may include and/or be included within one or more of the described components of system 400 of FIG. 4 and/or the systems/components of FIGS. 5A-8 and/or be used to perform operations described in correspondence to FIG. 9. The computing system 1000 is not intended to suggest any limitation as to scope of use or functionality of the present disclosure, as the innovations can be implemented in diverse general-purpose or special-purpose computing systems.

With reference to FIG. 10, computing environment 1010 includes one or more processing units 1022 and memory 1024. In FIG. 10, this basic configuration 1020 is included within a dashed line. Processing unit 1022 executes computer-executable instructions, such as for implementing any of the methods or objects described herein for detecting and/or indicating a position of a needle, or various other architectures, components, handlers, managers, modules, or services described herein. Processing unit 1022 can be a general-purpose central processing unit (CPU), a processor in an application-specific integrated circuit (ASIC), or any other type of processor. In a multi-processing system, multiple processing units execute computer-executable instructions to increase processing power. Computing environment 1010 can also include a graphics processing unit or co-processing unit 1030. Tangible memory 1024 can be volatile memory (e.g., registers, cache, or RAM), non-volatile memory (e.g., ROM, EEPROM, or flash memory), or some combination thereof, accessible by processing units 1022, 1030. The memory 1024 stores software 1080 implementing one or more innovations described herein, in the form of computer-executable instructions suitable for execution by the processing unit(s) 1022, 1030. The memory 1024 can also store needle position calculation data and/or raw data from an oscillator circuit or other data from elements of a detection circuit described herein, a composite graph data structure, including nodes, edges, and their respective attributes; a table or other data structure indicating states of a modeled system, configuration data, User Interface (UI) displays, browser code, data structures including data tables, working tables, change logs, output structures, input fields, output fields, data values, indices, or flags, as well as other operational data.

A computing system or environment 1010 can have additional features, such as one or more of storage 1040, input devices 1050, output devices 1060, or communication ports 1070. An interconnection mechanism (not shown) such as a bus, controller, or network interconnects the components of the computing environment 1010. Typically, operating system software (not shown) provides an operating environment for other software executing in the computing environment 1010, and coordinates activities of the components of the computing environment 1010.

The tangible storage 1040 can be removable or non-removable, and includes magnetic disks, magnetic tapes or cassettes, CD-ROMs, DVDs, or any other medium which can be used to store information in a non-transitory way and which can be accessed within the computing environment 1010. The storage 1040 stores instructions of the software 1080 (including instructions and/or data) implementing one or more innovations described herein.

The input device(s) 1050 can be a mechanical, touch-sensing, or proximity-sensing input device such as a keyboard, mouse, pen, touchscreen, trackball, a voice input device, a scanning device, or another device that provides input to the computing environment 1010. The output device(s) 1060 can be a display, printer, speaker, haptic feedback device, optical disk writer, or another device that provides output from the computing environment 1010.

The communication port(s) 1070 enable communication (e.g., wired and/or wireless communication) over a communication medium to another computing device. The communication medium conveys information such as computer-executable instructions or other data in a modulated data signal. A modulated data signal is a signal that has one or more of its characteristics set or changed in such a manner as to encode information in the signal. By way of example, and not limitation, communication media can use an electrical, optical, RF, acoustic, or other carrier.

In some examples, computer system 1000 can also include a computing cloud 1090 in which instructions implementing all or a portion of the disclosed technology are executed. Any combination of memory 1024, storage 1040, and computing cloud 1090 can be used to store software instructions and data of the disclosed technologies.

The present innovations can be described in the general context of computer-executable instructions, such as those included in program modules, being executed in a computing system on a target real or virtual processor. Generally, program modules or components include routines, programs, libraries, software objects, classes, components, data structures, etc. that perform tasks or implement particular abstract data types. The functionality of the program modules can be combined or split between program modules as desired in various embodiments. Computer-executable instructions for program modules can be executed within a local or distributed computing system.

The terms “system,” “environment,” and “device” are used interchangeably herein. Unless the context clearly indicates otherwise, none of these terms implies any limitation on a type of computing system, computing environment, or computing device. In general, a computing system, computing environment, or computing device can be local or distributed, and can include any combination of special-purpose hardware and/or general-purpose hardware and/or virtualized hardware, together with software implementing the functionality described herein. Virtual processors, virtual hardware, and virtualized devices are ultimately embodied in a hardware processor or another form of physical computer hardware, and thus include both software associated with virtualization and underlying hardware.

Physicians and nurses place millions of pIV catheters every day in order to administer life-saving medicine in a timely manner. Of those patients about 25% require multiple catheter placement attempts leading to increased pain for the patient, and increased cost associated with the procedure. The disclosed technology provides a solution to reduce the number of first time failures. Several different embodiments are presented herein (e.g., various needle-electrode designs and interface systems), although other designs are possible to implement the concept.

Embodiments of disclosed technology include can include one or more of the following features:
1) The catheter assembly is modular.
2) The detection unit can differentiate between different materials encountered when placing a catheter.
3) The system can effectively reduce the patient's pain and the cost associated with procedures.
4) The system assists the end user with catheter advancement by providing a robust feedback system including visual, audible, and/or tactile feedback of needle positioning/progression.

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.” As used herein, the term “coupled” generally means physically or chemically coupled or linked and does not exclude the presence of intermediate elements between the coupled items absent specific contrary language.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims and equivalents of the recited features. We therefore claim all that comes within the scope of the following claims.

1. A catheter insertion system, comprising:
a disposable catheter unit comprising a needle and a deployable catheter disposed over the needle;
a detection unit coupled to the catheter unit, the detection unit comprising circuitry electrically coupled to the needle and a skin electrode and configured to measure resistance between the needle and the skin electrode while the catheter unit is inserted or being prepared to be inserted into a patient and configured to determine an anatomical space within which an end portion of the catheter unit is located within the patient based on the measured resistance; and
an indicator interface configured to send control instructions to an output device to present an indication of the anatomical space within which the end portion of the catheter unit is located within the patient.

2. The system of claim 1, wherein the circuitry is configured to differentiate whether the end portion of the catheter unit is positioned in blood or in subcutaneous tissue based on the measured resistance.

3. The system of claim 1, wherein the anatomical space is mapped to an output state used to indicate a progress of the needle toward a target location.

4. The system of claim 3, wherein the target location is a vessel of the patient.

5. The system of claim 1, wherein the output device comprises a display.

6. The system of claim 1, wherein the output device comprises a haptic feedback device.

7. The system of claim 1, wherein the output device comprises a speaker system.

8. The system of claim 1, wherein the detection unit comprises a wrist-worn computing device, and wherein the skin electrode and an electrode on the needle is connected to an interface of the wrist-worn computing device.

9. The system of claim 1, wherein the detection unit is mounted on the skin electrode.

10. The system of claim 1, wherein the detection unit is mounted on the catheter unit.

11. The system of claim 1, wherein the output device comprises one or more of a display, a haptic feedback device, or a speaker included in the detection unit.

12. The system of claim 1, further comprising a wireless communication component configured to communicate additional control instructions to a second output device remote from the detection unit.

13. A method of indicating a position of a needle of a catheter system, the method comprising:
receiving a signal indicating a status of a progression of the needle toward a vessel of a patient;
determining an output corresponding to the indicated status; and
sending control instructions to an output device to present the output.

14. The method of claim 13, wherein the status is classified as one of a plurality of defined stages, and wherein each stage is mapped to a different output state.

15. The method of claim 13, wherein an output parameter is adjusted as a function of the status.

16. The method of claim 13, wherein the signal output by a detection unit based on signals from a skin electrode mounted on a skin of the patient and a needle electrode included in the needle, and wherein the detection unit comprises the output device.

17. The method of claim 13, further comprising presenting, via the output device, one or more of a visual indication, a haptic indication, and/or an audible indication of the indicated status.

18. An indicator system for a catheter insertion system, the indicator system comprising:
a detection unit comprising circuitry electrically coupled to a needle of a catheter unit and to a skin electrode and configured to measure resistance between the needle and the skin electrode while the catheter unit is inserted or being prepared to be inserted into a patient and configured to determine an anatomical space within which an end portion of the catheter unit is located within the patient based on the measured resistance; and
an output device configured to present an indication of the anatomical space within which the end portion of the catheter unit is located within the patient based on a signal generated by the detection unit.

19. The indicator system of claim 18, wherein the output device comprises a display, a haptic device, and/or a speaker configured to present the indication.

20. The indicator system of claim 18, wherein the indication indicates a status of a progression of the needle toward a vessel of a patient, and wherein an output parameter is adjusted as a function of the status, the output parameter comprising one or more of a volume or frequency of an audible output, an intensity or pattern of a haptic output, or a color or pattern of visual output.
